(19) United States
(12) Patent Application Publication
(10) Pub. No.: US 2003/0199760 A1
(43) Pub. Date: Oct. 23, 2003

(54) Device for permanently marking a selected tissue location within a patient ' s body

(75) Inventors: Navamanee Belinda Curpen, Toronto (CA); Claude Laflamme, Toronto (CA); Denise Ouimet-Oliva, Montreal (CA); Vincent Oliva, Ville Mont-Royal (CA)

Correspondence Address: OGILVY RENAULT, 1981 MCGILL COLLEGE AVENUE, SUITE 1600, MONTREAL, QC, H3A2Y3, CA

(73) Assignee: Mammoco Inc.

(21) Appl. No.: 10/400,413

(22) Filed: Mar. 28, 2003

Related U.S. Application Data
(63) Continuation of application No. PCT/CA01/01373, filed on Sep. 27, 2001.

(30) Foreign Application Priority Data
Sep. 29, 2000 (CA) 2,321,443

(51) Int. Cl. A61M 25/00

(52) U.S. Cl. 600/434

(57) ABSTRACT

A device for marking a selected tissue location within a patient's body comprises at least two markers (16, 116, 216) pre-loaded into a hollow needle (18, 118, 218) and packaged in a sterile fashion with a stopper (28, 128, 218) to prevent a stylet (27, 127, 227) inserted into the needle (18, 118, 218) from releasing the markers (16, 116, 216) during shipping. An indexing mechanism (28/29, 128/129, 228/229) is also provided for releasably blocking the stylet (27, 127, 227) at successive predetermined levels of insertion into the needle with each level of insertion being associated with the discharge of a single marker (16, 116, 216).

### RELATED APPLICATIONS

This is a continuation of International PCT application No. PCT/CA01/01373, filed on Sep. 27, 2001, which claims benefit of Canadian patent application No. 2,321,443.

### BACKGROUND OF THE INVENTION

1. Field of the Invention

The present invention relates to medical devices for localizing selected tissue sites in a body and, more particularly, to an improved device adapted to individually percutaneously deliver a selected number of permanent markers to desired tissue locations within a patient's body.

2. Description of the Prior Art

More and more, since the advent of breast screening program by mammography and extensive regulation of quality control, very small lesions are found before they become clinically palpable.

Since the eighties, there are technologies rendering these non-palpable lesions accessible for percutaneous biopsy, such as fine-needle aspiration biopsy, core biopsy or vacuum assisted biopsy. It is necessary to sample a portion of the lesion to obtain a diagnosis to prevent surgical excision in lesions that are benign and plan the best treatment in patients when cancerous cells are present. A minimum amount of tissue must be obtained to be able to have a representative sample of the lesion. In some cases, the lesions being biopsied are so small that after percutaneous biopsies, the lesions are either difficult to locate or completely removed.

Also, more and more, preoperative chemotherapy is administered after a diagnosis of breast cancer has been established by these percutaneous biopsies. After chemotherapy, the lesion completely disappears and there is a need to mark the precise location (the superior margin, the inferior margin, the lateral and medial margin) of the cancer so that the area can be properly excised.

Accordingly, various devices have been developed to enable relatively precise identification of a biopsy site for subsequent surgical procedures. For instance, U.S. Pat. No. 5,853,366 issued on Dec. 29, 1998 to Dowlatshahi discloses a tissue marking device comprising a hollow hypodermic needle having a distal piercing end adapted to be inserted into a patient's body to deliver a marker to a lesion site, using conventional imaging techniques. The marker is pushed out of the needle by a plunger mounted for free sliding motion with respect to the needle.

Although the tissue marking device disclosed in the above mentioned patent is effective, it has been found that there is a need for a tissue marking device adapted to safely and individually deliver a desired number of markers to selected tissue locations properly such that different biopsy sites can be distinguished from each other.

### SUMMARY OF THE INVENTION

It is therefore an aim of the present invention to provide a tissue-marking device adapted to individually deliver a desired number of markers without having to be reloaded.

It is also an aim of the present invention to provide a tissue-marking device which is relatively simple and economical to manufacture.

It is a further aim of the present invention to provide a tissue-marking device which can be conveniently used to deliver a desired number of markers to selected tissue locations.

Therefore, in accordance with the present invention, there is provided a kit for marking a selected tissue location within a patient's body, comprising an elongated guide member having a distal end adapted to be inserted into a patient's body to a selected tissue location, a proximal end extending outwardly from the patient's body once said distal end has been introduced therein, a passageway extending from said proximal end to said distal end, at least first and second markers adapted to be successively loaded into said passageway, and an actuator adapted to be inserted through said proximal end, into said passageway, to a first position in which further insertion of said actuator is temporally prevented so that a predetermined length of said actuator extends into said passageway to cause only said first marker to be discharged through said distal end, and from said first position to a second position in which a sufficient length of said actuator now extends into said passageway to cause said second marker to be discharged from said distal end, thereby ensuring individual discharge of said first and second markers.

In accordance with a further general aspect of the present invention, there is provided a kit for marking a selected tissue location within a patient's body, comprising an elongated guide member defining a passageway extending longitudinally therethrough between a distal end and a proximal end, said distal end being adapted to be inserted into a patient's body to a selected tissue location, at least two markers adapted to be successively loaded into said passageway, an actuator adapted to be inserted into said passageway, through said proximal end, for successively pushing said at least two markers out of said passageway through said distal end, and an indexing mechanism for releasably blocking said actuator at successive predetermined levels of insertion into said passageway, wherein each said predetermined level of insertion is associated with the individual discharge of one of said at least two markers.

### BRIEF DESCRIPTION OF THE DRAWINGS

Having thus generally described the nature of the invention, reference will now be made to the accompanying drawings, showing by way of illustration a preferred embodiment thereof, and in which:

FIG. 1 is a schematic, partly exploded, side view of a tissue-marking device in accordance with a first embodiment of the present invention;

FIG. 2 is a schematic perspective view of a stopper forming part of the device of FIG. 1;

FIG. 3 is a schematic, partly exploded, side view of a tissue-marking device in accordance with a second embodiment of the present invention;

FIG. 4 is enlarged side view of a proximal end portion of an actuator forming part of the device of FIG. 3;

FIG. 5 is a schematic side view of a tissue marking device in accordance with a third embodiment of the present invention; and

FIG. 6 is an enlarged perspective view of a proximal end portion of a stopper forming part of the device of FIG. 5.

### DESCRIPTION OF THE PREFERRED EMBODIMENTS

Now referring to the drawings, a device 10 for permanently marking a selected tissue location within a patient's body will be described.

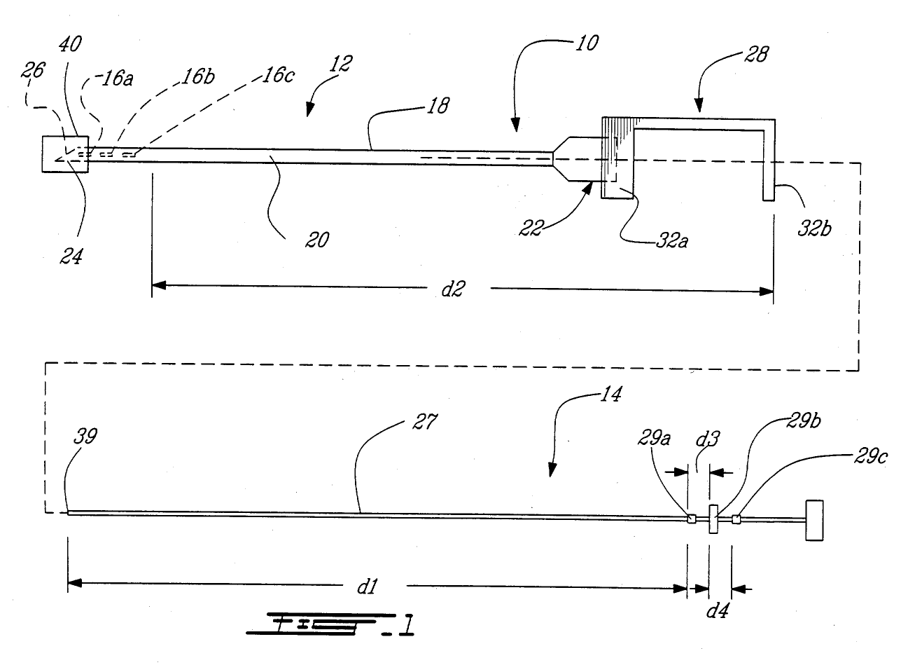

As shown in FIG. 1, the device 10 generally includes an elongated guide member 12 and an actuator 14 movable within the elongated guide member 12 in a predetermined sequence for individually releasing a certain number of markers 16a, 16b and 16c (three in the illustrated embodiment) that are pre-loaded into the elongated guide member 12.

The elongated guide member 12 can be provided in the form of a hollow needle or cannula 18 defining a passageway 20 extending longitudinally therethrough between a funnel-shaped proximal end 22 and a sharp distal end 24 defining an opening for percutaneously delivering the markers 16a, 16b and 16c to selected tissue locations within a patient's body, using conventional imaging systems. The distal end 24 is preferably beveled or angled to form a cutting edge 26 in order to facilitate the introduction thereof into the tissues of a patient's body, as is well known in the art. According to a preferred embodiment, the needle used is an 18-gauge spinal needle and is about 88 mm in length. It is understood that the dimensions of the needle 18 can vary depending upon the type and location of the site to be marked.

The markers 16a, 16b and 16c are preferably provided in the form of mechanically closed clips made of a biocompatible radiopaque non ferromagnetic material, such as titanium or the like, and have identical cross-sections which generally correspond to that of the passageway 20 to prevent the markers 16a, 16b and 16c from moving along the passageway 20 during manipulation of the needle 18. The markers 16a, 16b and 16c could also be made of metal, such as nitinol and other memory shaped alloys are contemplated as well. The markers 16a, 16b and 16c could also be made of radioactive material. The term radiopaque or non ferro magnetic or radioactive is herein intended to include any material that can be detected using conventional radiographic, sonographic or magnetic techniques or with a gamma probe. The markers 16a, 16b and 16c are adapted to be pre-loaded into the needle 18 through the proximal end 22 thereof down to the distal end 24. The markers 16a, 16b and 16c are configured to be wholly received within a patient's body and can have any appropriate anchoring means to prevent migration thereof after they have been delivered to a selected tissue location.

According to the illustrated embodiment, the actuator 14 is provided in the form of a stylet 27 properly sized to be slidably inserted into the passageway 20 through the proximal end 22 in order to successively pushed the markers 16a, 16b and 16c out of the distal end 24 of the needle 18.

As shown in FIG. 1, a stopper 28 is adapted to be mounted to the proximal end 22 of the needle 18 to cooperate with a predetermined number of spaced-apart index pins 29a, 29b and 29c (the number of index pins matching the number of markers) distributed along the proximal end portion of the stylet 27 and oriented in different radial directions so as to releasably block the stylet 27 at selected depths of insertion within the needle 18, as will be described hereinbelow.

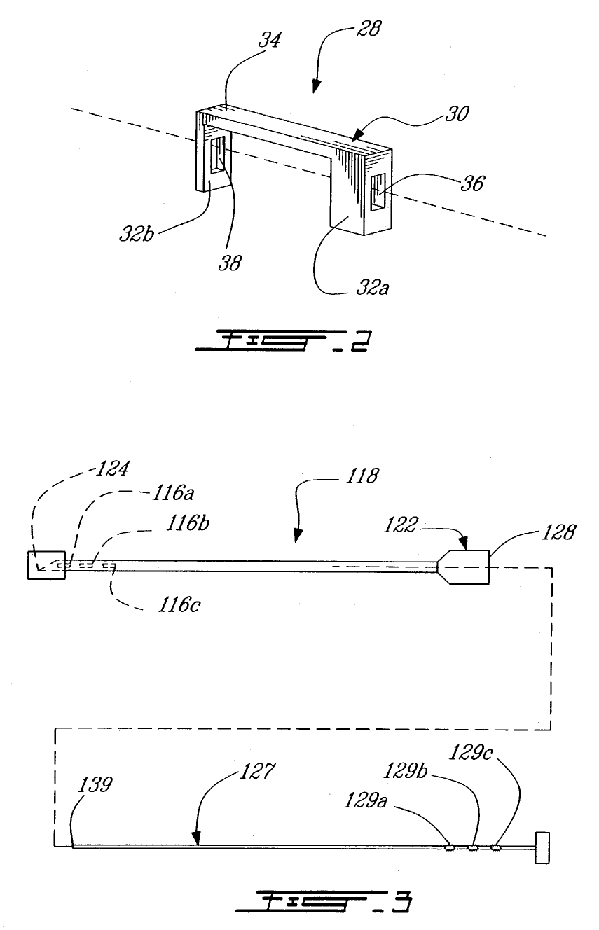

As shown in FIG. 2, the stopper 28 includes a substantially C-shaped body 30 having a pair of legs 32a and 32b spaced-apart by a web member 34. The distal leg 32a defines an opening 36 for allowing the stopper 28 to be tightly fitted over the proximal end 22 of the needle 18. The proximal leg 32b defines an opening 38 which is aligned with the opening 36 to form a passage for allowing the stylet 27 to be introduced into the passageway 20. The opening 38 is sized to successively receive the index pins 29a, 29b and ²9c when the same are respectively aligned therewith.

The distance d₁ between a leading end 39 of the stylet 27 and the first index pin 29a is selected to correspond to the distance d₂ between the trailing end of the third marker 16c and the outer surface of the leg 32b such that when the stylet 27 is introduced into the needle 18 through the stopper 28 with the first index pin 29a abutting against the outer surface of the leg 32b, the leading end 39 of the stylet 27 will be located immediately upstream of the third marker 16c.

To further insert the stylet 27 into the needle 18, the physician manipulating the device or needle has to rotate the stylet 27 about its longitudinal axis so as to angularly place the first index pin 29a in phase with the opening 38, thereby allowing the stylet 27 to freely slide into the needle 18 along a distance d3, that is until the second index pin 29b, which is 90 degrees offset with respect to the first index pin 29a, comes in contact with the outer surface of the leg 32b. The length of the stylet 27 that extends into the needle 18 when the second index pin 29b abuts the leg 32b is such that only the first marker 16a is pushed out of the distal end 24 of the needle 18 by the stylet 27. Indeed, the distance d₃ between the front face of the first index pin 29a and the second index pin 29b is equal to the respective length of each marker 16a, 16b and 16c.

When it is desired to discharge the second marker 16b, the physician has to rotate the stylet 27 90 degrees upon itself to place the second index pin 29b in phase with the opening 38, thereby allowing the stylet 27 to be further introduced into the needle 18 until the stylet stroke is blocked by the third index pin 29c, which is 90 degrees angularly offset relative to the second index pin 29b. The distance d₄ between respective front faces of the second and third index pins 29b and 29c also corresponds to a marker's length and, thus, only the second marker 16b will be ejected from the needle 18 during this second dispensing operation.

To eject the third marker 16c, the physician has to rotate the stylet 27 90 degrees upon itself so as to place the third index pin 29c in phase with the opening 38, and then pushed the stylet 27 further into the needle 18 to a fully inserted position thereof.

The above-described indexing mechanism advantageously prevents two or more markers from being inadvertently simultaneously expelled out of the needle 18 during a marking operation. Furthermore, the present invention advantageously allows to individually delivering a plurality of markers without having re-loading the device after each single marker delivery.

The needle 18, the markers 16a, 16b and 16c and the stylet 27 are preferably manufactured in the form of a kit wherein the markers 16a, 16b and 16c are pre-loaded into the needle 18 and packaged in a sterile fashion with a safety cap 40 fitted over the distal end 24 of the needle 18 and with the stopper 28 and the first index pin 29a cooperating to prevent the stylet 27 from releasing the markers 16a, 16b and 16c during shipping.

In use, the needle 18 is first inserted into the patient's body with the markers 16a, 16b and 16c loaded in the needle 18 so that when the distal end 24 thereof is located proximate to a selected tissue location, the stylet 27 can be manipulated as described hereinbefore to successively wholly position a selected number of markers 16 within the patient's body. A conventional visualization aid is used to confirm the position of the distal end 24.

The markers 16a, 16b and 16c can remain in the body even after the procedure is completed to allow subsequent identification and observation of the marked site(s). If desired, the markers 16a, 16b and 16c may be removed using conventional surgical techniques.

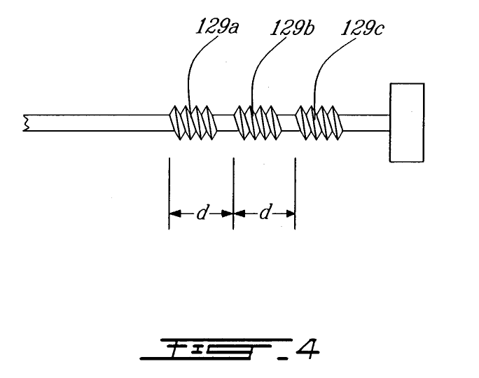

FIGS. 3 and 4 illustrate another embodiment of the present invention wherein the index pins 29a, 29b and 29c are replaced by three externally threaded zones 129a, 129b and 129c that are spaced-apart along a stylet 127 to successively threadably engage an internally threaded zone 128 provided in a proximal end 122 of a hollow needle 118 which is otherwise similar to needle 18. The distance between the beginning of successive threaded zones 129a, 129b and 129c is equal to the length of the pre-loaded markers 116a, 116b and 116c positioned at a distal end 124 of the needle 118.

The stylet 127 is sized to be freely slidable into the needle 118 until its first threaded zone 129a encounters the internally threaded zone 128 of the needle 118, thereby preventing the stylet 127 from being further slidably inserted into the needle 118. In this position a leading end 139 of the stylet 127 is located immediately adjacent the trailing end of the third marker 116c. To eject the first marker 116a, the physician must screw the stylet 127 so as to cause the first externally threaded zone 129a thereof to pass translativally beyond the internally threaded zone 128 of the needle 118, thereby allowing the stylet 127 to be subsequently further slidably inserted into the needle 118 until its second threaded zone 129b encounters the internally threaded zone 128 of the needle 118. The length of the stylet 127 which extends into the needle 118 when the second externally threaded zone 129b of the stylet 127 engages the internally threaded zone 128 of the needle 118 is such as to only cause the ejection of the first marker 116a.

The second marker 116b can be subsequently ejected by first screwing the stylet 127 into the needle 118 to cause the second externally threaded zone 129b of the stylet 127 to pass beyond the internally threaded zone 128 of the needle 118 and then pushing the stylet 127 further into the needle 118 until the third externally threaded zone 129c of the stylet 127 contacts the internally threaded zone 128 of the needle 118. The advancement of the stylet 127, i.e. the distance between the beginning of the second and third threaded zones 129b and 129c, corresponds to the length of the second marker 116b and, thus, cause the ejection thereof.

Finally, the third marker 116c can be discharged from the distal end 124 of the needle 118 by screwing the stylet 127 so as to cause the externally threaded zone 129c to enter into the needle 118 past the internally threaded zone 128 and subsequently pushing the stylet 127 further into the needle 118 to a fully inserted position thereof.

Other indexing mechanisms adapted to releasably block the stylet at different depths of insertion could be used as well.

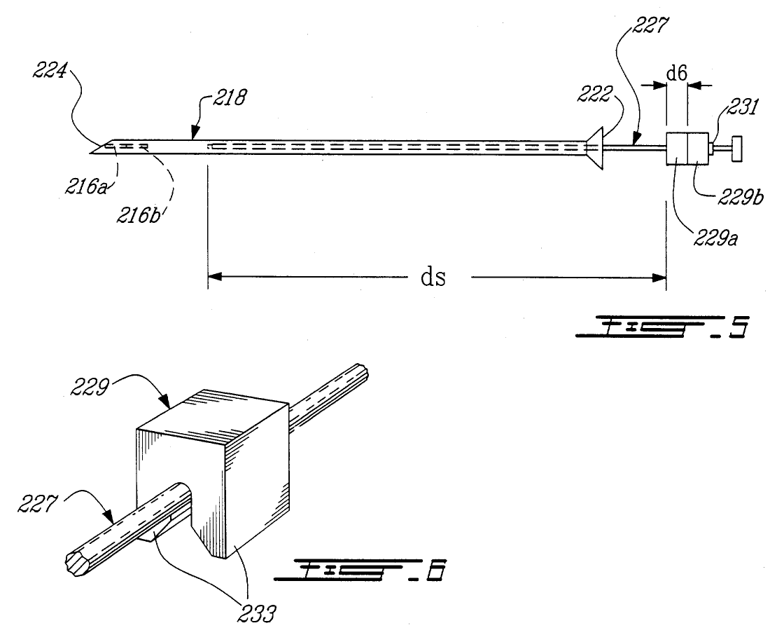

For instance, FIGS. 5 and 6 illustrate an indexing mechanism comprising a pair of stoppers 229a and 229b removably mounted at predetermined axial locations along a stylet 227 to limit the insertion thereof into a hollow needle 218 having a funnel-shaped proximal end 222 and a sharp distal end 224 loaded with a pair of markers 216a and 216b. The distance dₛ between the leading end of the stylet 227 and the front surface of the first stopper 229a corresponds to the length required to abut the trailing end of the second marker 216b. The distance d₆ between the front surfaces of the stoppers 229a and 229b is equal to the length of the markers 216a and 216b. The front surfaces of the stoppers 229a and 229b are configured to abut against the proximal end 222 of the needle 218 to prevent further insertion of the stylet 227 into the needle 218.

As shown in FIG. 5, a reference abutment surface 231 can be fixed on the stylet 227 to ensure proper positioning of the removable stoppers 229a and 229b.

As shown in FIG. 6, each stopper 229 can be provided in the form of a block having a pair of resilient legs 233 adapted to tightly grip the stylet 227 therebetween to provide a snap-fit engagement of the stopper on the stylet 227. In this way, the stoppers 229a and 229b can be readily manually withdrawn from the stylet 227 by simply pulling on the stoppers 229a and 229b so as to cause the legs 233 thereof to be deflected laterally outwardly.

To eject the first marker 216a from the distal end 224 of the needle 218, the first stopper 229a is removed from the stylet 227 and the stylet 227 is inserted into the needle 218 until the front surface of the second stopper 229b engages the proximal end 222 of the needle 218. Then, to eject the second marker 216b, the second stopper 229b is removed and the stylet 227 is further pushed into the needle 218, thereby causing the second marker to be ejected out of the leading end 224 of the needle 218.

When it is desired to deliver the two markers 216a and 216b at one location, the two stoppers 229a and 229b are removed to then allow the stylet 227 to be fully inserted into the needle 218 in a single step.

1. A kit for marking selected tissue locations within a patient's body, comprising an elongated guide member defining a longitudinal passageway extending between a distal end and a proximal end of said elongated guide member, said distal end being adapted to be inserted into a patient's body to selected tissue locations, at least two markers adapted to be successively loaded into said passageway, an actuator adapted to be inserted into said passageway, through said proximal end, for successively pushing said at least two markers out of said passageway through said distal end, and an indexing mechanism for releasably blocking said actuator at successive predetermined levels of insertion into said passageway, wherein each said predetermined level of insertion is associated with the individual discharge of one of said at least two markers.

2. A kit as defined in claim 1, wherein said indexing mechanism includes first and second indexing parts respectively provided on said actuator and said elongated guide member, said first and second indexing parts cooperating together for allowing said actuator to be incrementally inserted into said elongated guide member between axially spaced-apart index positions.

3. A kit as defined in claim 2, wherein said first indexing part includes at least two axially disposed indexing members, and wherein said second indexing part includes a stopper defining an opening configured to receive the indexing members when aligned therewith, said indexing members being angularly offset with respect to one another so that when a first one of said indexing member is in alignment with said opening, a second adjacent one of said indexing members is angularly out of phase with said opening, thereby preventing further advancement of said actuator into said elongated guide member.

4. A kit as defined in claim 3, wherein said actuator is rotatable about a longitudinal axis thereof while extending within said elongated guide member for allowing said second one of said indexing members to be brought in phase with said opening in order to permit further advancement of said actuator into said elongated guide member.

5. A kit as defined in claim 2, wherein said second indexing part includes an internally threaded zone provided in said passageway of said elongated guide member, and wherein said first indexing part includes at least two successive externally threaded zones axially spaced-apart along the actuator for successively threadably engaging said internally threaded zone.

6. A kit as defined in claim 5, wherein respective leading ends of said successive externally threaded zones are spaced by a distance corresponding to a length of said markers.

7. A kit as defined in claim 1, wherein said indexing mechanism includes at least one indexing member mounted at a predetermined axial location along said actuator to act as a stopper for solely allowing a desired length of said actuator to extend into said passageway, said indexing member being removable from said actuator for allowing further insertion of said actuator into said passageway.

8. A kit as defined in claim 1, wherein said passageway and said markers are dimensioned so as to provide a snug fit to prevent movements of said markers in the absence of a pushing action of said actuator.

9. A kit as defined in claim 1, wherein said markers are pre-loaded within said elongated guide member, and wherein a safety device is provided to prevent said markers from being inadvertently released from said elongated guide member.

10. A kit as defined in claim 1, wherein said actuator is pre-inserted into said passageway so that a distal end thereof is located immediately upstream of said markers.

11. A kit as defined in claim 1, wherein said elongated guide member and said actuator are packaged in a sterile fashion with a stopper to prevent said actuator from being inadvertently actuated and so release said markers during shipping.

12. A device for delivering markers to selected tissue locations within a patient's body, comprising an elongated guide member having a distal end adapted to be inserted into a patient's body to selected tissue locations, a proximal end extending outwardly from the patient's body once said distal end has been introduced therein, a passageway extending from said proximal end to said distal end, and an actuator adapted to be inserted through said proximal end, into said passageway, to a first position in which further insertion of said actuator is temporarily prevented so that only a predetermined length of said actuator extends into said passageway to cause only one marker to be discharged through said distal end, and from said first position to a second position in which a sufficient length of said actuator now extends into said passageway to cause a second marker to be discharged from said distal end, thereby ensuring individual discharge of the markers.

13. A device as defined in claim 12, wherein the markers are pre-loaded within said elongated guide member, and wherein a safety device is provided to prevent said markers from being inadvertently released from said elongated guide member.

14. A device as defined in claim 13, wherein said safety device includes a safety cap adapted to be fitted over said distal end.

15. A device as defined in claim 14, wherein said safety device further includes a stopper to prevent said actuator from being inadvertently further pushed into said passageway.

16. A device as defined in claim 12, wherein said actuator is pre-inserted into said passageway so that a distal end thereof is located immediately upstream of said markers.

17. A device as defined in claim 12, wherein said elongated guide member and said actuator are packaged in a sterile fashion with a stopper to prevent said actuator from being inadvertently actuated and so release said markers during shipping.
